(19) United States
(12) Patent Application Publication
(10) Pub. No.: US 2002/0133058 A1
(43) Pub. Date: Sep. 19, 2002

(54) Prophylactic cover and drape for endoscopic camera system

(76) Inventor: Mitchell C. Calderwood, Goleta, CA (US)

Correspondence Address: Sanford J. Piltch, 2895 Hamilton Blvd., The Atrium - Suite 204, Allentown, PA, 18104, US

(21) Appl. No.: 09/809,505

(22) Filed: Mar. 15, 2001

Related U.S. Application Data
(60) Provisional application No. 60/235,652, filed on Sep. 26, 2000.

(51) Int. Cl. A61B 1/04

(52) U.S. Cl. 600/122

(57) ABSTRACT

A sterile barrier, cover or drape for use with surgical endoscopic camera systems, or the like, which utilizes an optically clear elastomeric film mounted across the aperture of cooperating disks press-fit together to clamp the film and the drape between the disks for separation of the scope and the camera lens while maintaining a sterile field separation between the camera and the mating scope during exploratory and surgical procedures

### BACKGROUND OF THE INVENTION

The present invention deals with a cover and drape, i.e. a barrier system, between a flexible insertable imaging tool which is insertable into and removable from a human orifice such that bacteria and other particulate and liquid matter can be kept away from the surfaces of the tool while maintaining clarity of image to the user. More particularly the invention provides an optically transparent elastomeric cover between the camera lens system and an endoscope or laprascope, or the like with an attached barrier drape which extends away from the junction between the surgical scope and the camera lens system for the purpose of covering the camera and the cabling for a certain distance to prevent contamination of the camera system by any bodily fluids or other tissues which may carry bacteria, virus or other contaminants.

The invention deals with a “closed camera drape” which forms a barrier for the camera (and lens) and continues for a certain distance along the cabling of a camera system used for invasive imagery of body organs through one or more orifices. The barrier of the present invention is positioned between the flexible (or non-flexible) insertable imaging tool and the camera lens and body. There currently is available a transparent hard disk coupling system which disk is molded into an outer ring which is insertable between the “scope” and the camera body and lens. The ring is heat sealed to an accordion-like drape which extends rearward away from the “scope” and over the cables for a short distance. This system is not very popular as the ring diameter and the lens diameter usually do not match and mounting becomes difficult, if not impossible in some instances. Further, it is often the case that as the hard disk is being mounted to the camera that a crack or break occurs which defeats the barrier and sometimes even damages the camera or lens.

The barrier of the present invention cures the problem of mounting to several differing diameters by providing an optically clear elastomeric film installed across the aperture of an oversized ring which is overlaid upon the lens system. The “scope” can then be positioned into the lens mounting system with the film stretching to provide the attachment of the “scope” to the camera with the film remaining as a barrier between. The internal diameter of the ring will be just slightly larger than the largest scope attachment which is available.

The optically clear elastomeric film is reusable within a single procedure and resists tearing, while stretching to accommodate the insertion and mount coupling of the scope to the camera lens and body. Mount coupling is accomplished by either a press-fit into a ring with a detent release spring or a bayonet partial turn into the camera lens. Neither mount coupling has an adverse effect on the integrity of the film.

The present invention provides a clear advantage over currently available sterile barrier systems by permitting the barrier system to be mount coupled to any of several different camera systems having a wide variety of lens coupling systems and diameters of lenses. Also, cold sterilization of the camera head or body, which reduces camera life, is avoided. Cold sterilization of the camera, i.e., cold soaking in sterilizing solutions such as glutereldahydes, reduce camera life by approximately one-third and require significant time periods in which to complete the sterilization process. One must note that heat or steam sterilization procedures are not available as these procedures tend to destroy the cameras. Thus, unless some type of barrier between the scope and the camera is utilized during a surgical procedure, taking into account the time required for cold sterilization, a significant number of cameras will be required for use in operating room environments.

It is also known that surgeons tend to utilize a variety of scopes during a single procedure which, without a barrier between the scope(s) and the camera, will tend to contaminate the camera requiring sterilization. The exchange of two or more scopes with the camera would normally require the changing of the barrier. However, the sterile barrier system of the present invention provides for repeated use without tearing, as the elastomeric film utilized across the ring resists tearing while stretching to accommodate the mount coupling of the scope.

It is, therefore, an object of the present invention to provide a tear resistant, elastomeric film, which is optically transparent, for use in creating a barrier for imaging of internal organs and cavities of human using one or more scope arrangements attached to a camera imaging system.

It is also an object of the present invention to extend a second barrier rearward over the camera body, and over the cabling, a distance sufficient to prevent contamination of difficult to clean surfaces, or surfaces which are difficult to disinfect.

It is a further object of the present invention to provide a universal mount coupling system using the elastomeric film as the coupling device so that all camera imaging systems and their matable scopes can use the sterile barrier.

Other objects will appear hereinafter.

### SUMMARY OF THE INVENTION

The present invention may be described as a sterile barrier cover or drape for use with surgical endoscopic camera systems, or the like, which utilizes an optically clear elastomeric film for separation of the scope and the camera lens while maintaining a sterile field separation between the camera and the mating scope during exploratory and surgical procedures. The retention of the sterile field eliminates the need for subsequent cold sterilization of the camera “head” (lens and body), as well as the intervating cabling. Further, the inherent abrasion and tear resistance of the film barrier allows for the interchanging of several scopes during a single procedure without puncturing the film barrier, or tearing the film, when mount coupling or decoupling a scope.

The present invention may be described in further detail as a disposable cover and drape for an endoscopic camera system having an optically clear elastomeric film mounted across the annular opening of a first ring segment which ring segment is insertable between an elongate lens assembly for insertion into a human body cavity and a mating camera coupler. The first ring segment is appropriately dimensioned to cooperatively mate with and be secured to a second ring segment carrying an expandable flexible plastic tube having a first open end mounted to the annular opening of the second ring segment and a second open end for expanding over and substantially covering the camera system for preventing contamination of the outer surfaces of said camera system in conjunction with a surgical or exploratory procedure. The elastomeric film separating the camera coupler and the elongate lens has sufficient elastic memory to accept repeated mounting and de-mounting of one or more elongate lens assemblies without tearing, cracking, splitting or rupturing such that said elastomeric film remains intact and free of distortion. An alternate embodiment of the invention includes a third ring segment positioned intermediate the first and second ring segments such that each of the ring segments cooperatively mates with a third ring segment.

The optically clear elastomeric film may be made from a material selected from a group of elastomeric urethanes: including polyether or polyester based aliphatic, polycaprolactate aliphatic, cycloaliphatic or aromatic, or any blend thereof, or may be made from elastomeric silicones. The optically clear elastomeric film also has certain physical characteristics and properties which permit clear visual acuity through the film regardless of the exchange of elongate. Further, the expandable flexible plastic tube may be made from any waterproof elastomeric or plastic material having a flexibility to collapse and extend over the camera system and associated cabling to protect the camera system from contamination.

### BRIEF DESCRIPTION OF THE DRAWINGS

For the purpose of illustrating the invention, there is shown in the drawings forms which are presently preferred; it being understood, however, that the invention is not limited to the precise arrangements and instrumentalities shown.

FIG. 1 is a side plan view of the cover and drape of the present invention.

FIG. 2 is an exploded view of the cover and drape of the present invention showing the intervening elastomeric film barrier and mounting ring.

FIG. 3 is a perspective view of the elastomeric film barrier, mounting ring and drape of the present invention.

FIG. 4 is a sectional view taken along Line 4-4 of FIG. 3.

### DETAILED DESCRIPTION OF THE PREFERRED EMBODIMENTS

The following detailed description is of the best presently contemplated mode of carrying out the invention. The description is not intended in a limiting sense, and is made solely for the purpose of illustrating the general principles of the invention. The various features and advantages of the present invention may be more readily understood with reference to the following detailed description taken in conjunction with the accompanying drawings.

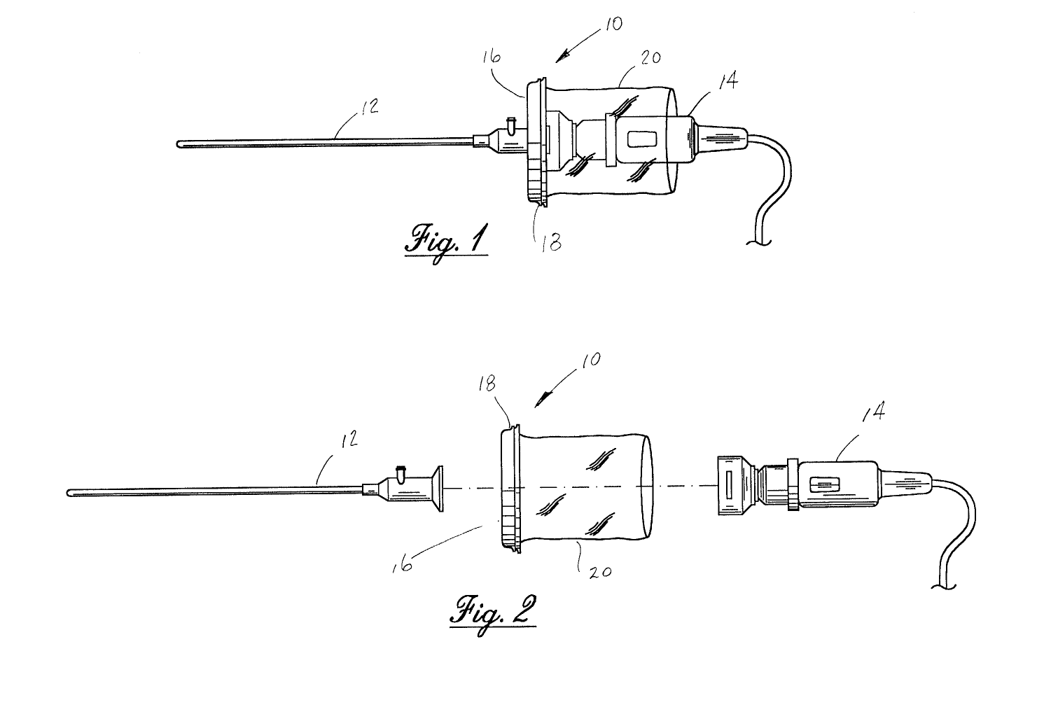

Referring now to the drawings in detail, where like numerals refer to like parts or elements, there is shown in FIG. 1 the sterile barrier 10 of the present invention interposed between an endoscope 12 and a camera imaging system 14. One can better see the individual elements of the barrier 10 by reference to FIG. 2. The barrier 10 has three principle parts. These parts are the flexible, optically transparent, tear resistant elastomeric film 16 which is stretched across the opening and between a pair of matable cooperating ring segments 18 and a distending drape of a flexible plastic material 20. The ring segments 18 retain the film in a stretched condition across the internal aperture of the ring segments 18. See FIG. 4. The elastomeric film 16 forms a sterile barrier between the endoscope (or other “scope”) 12 and the camera imaging system 14, as well as serving as the coupler device for supporting and holding the ring segments 18 and drape 20 in position shielding the camera lens, body and cabling from contamination.

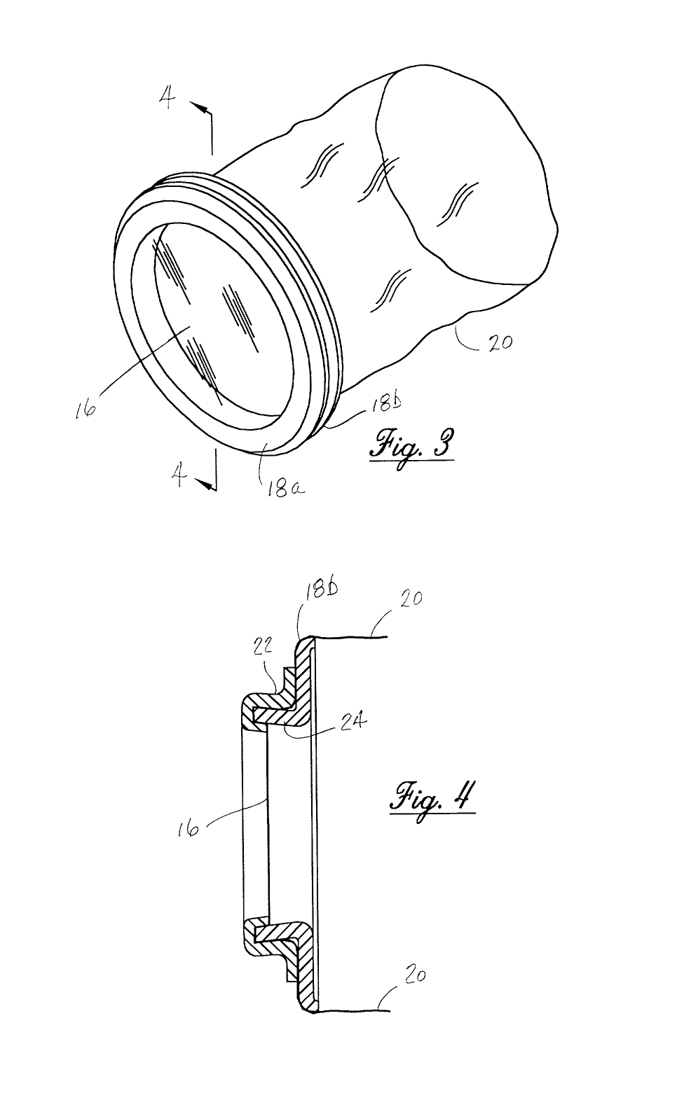

The optically transparent elastomeric film 16 is held tautly across the aperture of the ring segments 18a, 18b and is clamped between the cooperatively mating portions on the juxtaposed periphery of the ring segments as shown in FIG. 4. The drape portion 20 of the barrier 10, which covers the camera 14 is clamped within the same cooperatively mating portions of the ring segments 18a, 18b. The elastomeric film 16 is die cut to a circle slightly larger than the aperture of the ring segments 18a, 18b. The drape portion 20 is formed as a tube tapering to an opening also slightly larger than the aperture of the ring segments 18a, 18b. Both the elastomeric film 16 and the drape portion 20 of the barrier 10 are positioned between the ring segments 18a, 18b and the rings are press-fit together such that the flange 22 of ring segment 18a captures both the elastomeric film 16 and the drape 20 against the collar 24 of ring segment 18b. The drape portion 20 of the barrier 10 can also be attached to ring segment 18b by affixing the peripheral edge of the opening of the elongated tapered tube to the back of ring segment 18b as shown in FIG. 3. In this manner the drape portion 20 extends away from the scope 12 and along and over the camera imaging system 14 from the outer circumference of the ring segment 18b.

The elastomeric film 16 can also be attached to ring segment 18a by affixing the peripheral edge of the film 16 to the front of the ring segment 18a. In this manner the elastomeric film 16 still provides a covering or barrier across the lens of the camera system 14 The ring segments 18a, 18b cooperatively act to join the film 16 (covering the front (lens) of the camera system) with the drape 20 (covering the remainder of the camera system) to form a sterile barrier 10 over the entire camera system. Elastomeric film 16 also provides both an optically transparent lens between the “scope” and the camera lens and a base for coupling the barrier 10 to the combined scope/camera system.

Other methods of attaching the elastomeric film 16 and the drape 20 to the ring segments 18 are to be considered as part of the present invention. These methods include, but are not limited to, heat sealing, ultrasonic welding, all manners of adhesive attachment, or any other means of attaching the two ring segments together, or to an intermediary ring.

The optically clear elastomeric film 16 may be selected from one of the following types of elastomeric urethanes: polyether or polyester based aliphatic, polycaprolactate aliphatic, cycloaliphatic or aromatic, or any blend thereof, and certain elastomeric silicones. The elastomeric film 16 should exhibit the following physical characteristics. It is recommended to have an elongation or elasticity factor of between 100% to 1000%, a hardness over the range of 50 Shore A to 50 Shore D, a modulus of 1.0 to 15.0 MPa at 100% elongation and 2.0 to 50.0 at 300% elongation, and exhibit a resistance to tearing or abrading with a clarity of optically transparent to provide optimum visual acuity. The drape 20 may be selected from any waterproof elastomeric or plastic material having a flexibility to collapse and extend in accordion-like fashion and have a thickness in the range of 1 mil to 5 mils.

When the barrier 10 is utilized, the camera system 14 with its associated cabling is inserted into the drape 20 which has already been attached to the elastomeric film 16 by utilizing ring segments 18a, 18b in accordance with the foregoing description. Then, with the elastomeric film 16 positioned in front of or against the lens of the camera 14, and with the barrier 10 held in place, the scope 12 is positioned on the opposite side of the film 16 as shown in FIG. 2. In this drawing, the coupling mount between the scope 12 and the camera 14 is internal such that the scope 12 is pushed inward extending the film 16 down and into the mount of the camera lens. The scope 12 may be secured either by a bayonet twist coupling or by a spring release detent and clamp, either can be used without tearing or rupturing the film 16. In the event that a different scope 12 needs to be utilized, the uncoupling is accomplished by reversing the process and mounting the different scope 12 to the camera system 14, but without the necessity of replacing the sterile barrier 10. The elastomeric film 16 accommodates the elongation of the distance necessary for the mounting member of the scope 12 to extend into, or over, the camera lens mount so that a coupling can occur, all without tear, crack, split or rupture so that the barrier 10 remains intact and free of wrinkles which could cause visual distortion. Once the barrier 10 is in position between the scope 12 and the camera system 14, the drape 20 can then be extended over the camera system 14 and its cables to protect them from any unwanted contamination.

In this manner the present invention provides a coupling base for the connection of the scope 12 and camera imaging system 14, without the need for additional mounts and coupling apparatus, and a structural base for the extension of the drape 20 over the remaining portions of the camera imaging system 14 behind the lens mount. Further, the present invention also provides for the continuing integrity of the barrier 10 even if an interchange of scopes 12 may be necessary, without any compromise of the sterile barrier 10 by a rupture in the film 16 compromising the sterile field and exposing complex optical and electronic camera parts to the need for sterilization.

The present invention may be embodied in other specific forms without departing from the spirit or essential attributes thereof and, accordingly, the described embodiments are to be considered in all respects as being illustrative and not restrictive, with the scope of the invention being indicated by the appended claims, rather than the foregoing detailed description, as indicating the scope of the invention as well as all modifications which may fall within a range of equivalency which are also intended to be embraced therein.

1. A disposable cover and drape for an endoscopic camera system comprising an optically clear elastomeric film mounted across the annular opening of a first ring segment and insertable between an elongate lens assembly for insertion into a human body cavity and a mating camera coupler, said first ring segment being dimensioned to cooperatively mate with and be secured to a second ring segment carrying an expandable flexible plastic tube having a first open end mounted to the annular opening of the second ring segment and a second open end for expanding over and substantially covering said camera system for preventing contamination of the outer surfaces of said camera system in conjunction with a surgical or exploratory procedure, said elastomeric film having sufficient elastic memory to accept repeated mounting and de-mounting of one or more elongate lens assemblies without tearing, cracking, splitting or rupturing such that said elastomeric film remains intact and free of distortion.

2. In accordance with claim 1, wherein each of the ring segments cooperatively mates with a third ring segment positioned intermediate the first and second ring segments.

3. In accordance with claim 1, said optically clear elastomeric film may be made from a material selected from a group of elastomeric urethanes including polyether or polyester based aliphatic, polycaprolactate aliphatic, cycloaliphatic or aromatic, or any blend thereof.

4. In accordance with claim 1, said optically clear elastomeric film may be made from elastomeric silicones.

5. In accordance with claim 1, said optically clear elastomeric film having an elongation factor of between 100 percent to 1000 percent.

6. In accordance with claim 1, said optically clear elastomeric film having a hardness over the range of 50 Shore A to 50 Shore D.

7. In accordance with claim 1, said optically clear elastomeric film having a modulus of 1.0 to 15.0 MPa at 100% elongation and a modulus of 2.0 to 50.0 MPa at 300% elongation.

8. In accordance with claim 1, said expandable flexible plastic tube may be made from any waterproof elastomeric or plastic material having a flexibility to collapse and extend in accordion-like fashion with a material thickness in the range between 1.0 mil to 5.0 mils.
